(19) United States
(12) Patent Application Publication
(10) Pub. No.: US 2005/0079123 A1
(43) Pub. Date: Apr. 14, 2005

(54) Method and apparatus for microbial decontamination

(76) Inventor: Robert K. Shuler, Houston, TX (US)

Correspondence Address: WONG, CABELLO, LUTSCH, RUTHERFORD & BRUCCULERI,;P.C., 20333 SH 249, SUITE 600, HOUSTON, TX, 77070, US

(21) Appl. No.: 10/946,397

(22) Filed: Sep. 21, 2004

Related U.S. Application Data
(60) Provisional application No. 60/510,801, filed on Oct. 10, 2003.

(51) Int. Cl. A61K 33/14; C01B 11/02

(52) U.S. Cl. 423/477; 424/661

(57) ABSTRACT

A process and apparatus is provided for generating chlorine dioxide gas for the fumigation of enclosed spaces that includes adding reactants for generation of chlorine dioxide by dropwise addition to an aqueous reaction medium in a sealed reaction chamber while bubbling a motive gas through the aqueous reaction medium, whereby the motive gas releases the generated chlorine dioxide gas from the aqueous reaction medium and drives the release of chlorine dioxide gas from reaction chamber.

### CROSS-REFERENCE TO RELATED APPLICATION

This application claims the benefit of U.S. Provisional Application No. 60/510,801 filed Oct. 10, 2003.

### FIELD OF THE INVENTION

The invention relates to apparatus and methods for microbial fumigation of enclosures using chlorine dioxide gas.

### BACKGROUND OF THE INVENTION

Chlorine dioxide (ClO₂) is a recognized antimicrobial, or microbicidal, agent that has been used for disinfection since the early 1900s. Chlorine dioxide is an effective bactericidal agent in parts-per-million concentrations and has been shown to have sporicidal and virucidal activity as well.

Chlorine dioxide can be generated in a gas or liquid form. However, chlorine dioxide is unstable as a gas and decomposes rapidly to form chlorine gas (Cl₂), oxygen (O₂) and heat. At concentrations over about 10% (300 mg/liter) chlorine dioxide gas may decompose explosively. For these reasons, chlorine dioxide gas cannot be safely stored and is thus generated on site or provided as a stabilized aqueous solution such as the PUROGENE brand of 2% aqueous stabilized chlorine dioxide. For on-site generation, chlorine dioxide gas is typically either generated in aqueous solution such as by electrochemical oxidation or is rapidly dissolved in water as soon as it is generated. Chlorine dioxide is soluble and relatively stable in aqueous solution.

In 1967, EPA first registered the liquid form of chlorine dioxide for use as a disinfectant and sanitizer for use at a variety of sites including farm animals, bottling plants, food processing, handling and storage plants, and many others. Stabilized liquid chlorine dioxide is applied to hard surfaces with a sponge or mop or as a coarse spray.

For food processing the FDA only permits the generation of chlorine dioxide in water by treating an aqueous solution of sodium chlorite with either chlorine gas or a mixture of sodium hypochlorite and hydrochloric acid, or treating an aqueous solution of sodium chlorate with hydrogen peroxide in the presence of sulfuric acid. The concentration of chlorine dioxide in water cannot exceed 3 ppm residual chlorine dioxide.

The EPA first registered chlorine dioxide gas as an antimicrobial pesticide in the 1980s. Chlorine dioxide gas is registered for sterilizing manufacturing and laboratory equipment, environmental surfaces, tools, and clean rooms. For example, Rosenblatt et al (U.S. Pat. No. 4,681,739) teach a method for sterilizing microbiologically contaminated articles, such as the dry and gas impermeable surfaces of medical or dental implements or other articles contaminated with live bacteria and bacterial spores, using chlorine dioxide gas. In this method, the medical instruments are placed in a partially evacuated chamber which is subsequently humidified. Chlorine dioxide gas is generated locally and introduced into the chamber where it is held for a desired period of time. The chlorine dioxide gas for use in this method can be generated by a variety of means including passing a stream of chlorine gas diluted with air or nitrogen at a metered rate through a column of finely divided sodium chlorite, or treating a dilute solution of aqueous potassium persulfate with a dilute solution of aqueous sodium chlorite at ambient temperatures in a closed reaction vessel.

Chlorine dioxide formulations have many other industrial uses including: bleaching pulp, paper and textiles, washing fruit and vegetables, disinfecting flume water, disinfecting meat and poultry, disinfecting food processing equipment, sanitizing water, controlling odors, treating medical wastes and treating municipal water.

Essentially three different categories of chemistries are employed for producing commercial quantities of ClO₂: electrolysis of chlorite, reduction of the chlorate ion (ClO₃⁻) and oxidation of the chlorite ion (ClO₂⁻). One example of an electrolytic process is taught by Twardowski et al in U.S. Pat. No. 4,683,039 and is embodied in an apparatus commercially available from ERCO Worldwide of Toronto, Ontario, Canada. In this method, electrochemical oxidation of the chlorite ion in an aqueous sodium chlorite (NaClO₂) solution is followed by separation of the generated chlorine dioxide in aqueous solution across a gas pore membrane. The products of the reaction are the desired ClO₂ in addition to caustic soda and hydrogen gas as depicted by the following reaction scheme: NaClO₂+H₂O→ClO₂+NaOH+½ H₂. This apparatus and method produces ClO₂ in aqueous solution primarily for pulp, paper and water treatment.

A number of different reducing agents can be used in the reduction of chlorate including sulfur dioxide (SO₂), methanol (CH₃OH), chloride ion (Cl⁻), and hydrogen peroxide (H₂O₂). In commercial practice the reduction reaction employs sodium chlorate (NaClO₃) in reaction with either sulfuric (H₂SO₄) or hydrochloric acid (HCl). In one method for the generation of liquid ClO₂ for the pulp and paper industry, sodium chlorate (NaClO₃) is reacted with hydrochloric acid (HCl) in gaseous form to generate chlorine dioxide in titanium coated generators. Alternatively, Mason et al (U.S. Pat. No. 5,204,081) teach a vacuum driven mixing method that utilizes a water venturi to pull sodium chlorate (NaClO₃) into mixture with either anhydrous hydrogen chloride or hydrochloric acid to form ClO₂ in gaseous form immediately prior to water entrainment.

Similarly, several chemistries are available for the oxidation of the chlorite ion to generate chlorine dioxide. A commonly used chemistry relies on the reaction of sodium chlorite (NaClO₂) with chlorine (Cl₂) gas via gaseous chlorination (Cl₂+NaClO₂→2ClO₂+NaCl). The mixing of chlorine dioxide with chlorine gas can be performed by a variety of methods as embodied in various commercial generators including the ERCO R102 Generator from Sterling Pulp Chemicals. An apparatus available from Sabre Oxidation Technologies, Inc. embodies a vacuum driven mixing method as taught by Mason et al (U.S. Pat. No. 6,468,479) that utilizes a water venturi eductor to pull sodium chlorite (NaClO₂) into mixture with chlorine gas (Cl₂) to form ClO₂ in gaseous form immediately prior to water entrainment. However, the requirement for chlorine gas is a disadvantage of all of these methods.

For generation of chlorine dioxide gas for gas fumigation purposes, several manufacturers such as CDG and ClorDiSys Solutions have developed specialized solid phase gaseous chlorination systems that use premixed cylinders of compressed chlorine-in-nitrogen gas for interaction over reactor cartridges packed with thermally stable solid sodium chlorite. In addition to the requirement for chlorine gas, this system requires special sodium chlorite compositions that are provided in prepacked canisters.

Alternatively, sodium chlorite can be reacted with hydrochloric acid to form chlorine dioxide in the general reaction: NaClO₂⁻+HCl⁺→ClO₂+NaCl+H₂O. Commercially available generators using this reaction are limited in the amount of chlorine dioxide they can safely produce and involve sophisticated metering electronics. The chlorine dioxide produced is immediately entrained in a motive water stream, which is thereby sterilized. One commercial generator employing this technology is the BELLO ZON generating plant available from ProMinent Fluid Controls, Inc. Chemical generators are also available that utilize the reaction of three chemicals, sodium chlorite, hydrochloric acid and sodium hypochlorate (bleach). All of these apparatus are sophisticated, expensive and are adapted for treating fluids such as water or oil into which the chlorine dioxide is mixed as it is generated.

On the basis of its effectiveness and years of application as a disinfectant/sanitizer, chlorine dioxide was selected for treatment of the US Postal Service building contaminated with anthrax spores in late 2001-2002. However, despite its many prior uses, an expert in the field testified to Congress on the absence of information published in the refereed scientific literature on the use of chlorine dioxide gas for the disinfection of large buildings or spaces. For this reason, considerable effort was put into the development of a custom built on-site chlorine dioxide gas generation plant for this decontamination effort. The reactants for generation of chlorine dioxide gas included sodium chlorite, sodium hypochlorite, and hydrochloric acid. It is believed that the chlorine dioxide gas produced was immediately entrained in water which was then pumped over a packed bed from which the chlorine dioxide gas was released by forced air. The cost of this effort was in the tens of millions of dollars.

There remains a need for simple inexpensive apparatus and methods for the microbial fumigation of rooms, large containers, dwelling sized spaces and buildings. One such purpose is mold remediation. Mold contamination in buildings has become a significant economic problem particularly in the South and in areas experiencing flooding. Molds can trigger asthma episodes in sensitive individuals with asthma and can trigger allergies in sensitive individuals. Common indoor molds are Cladosporium, Penicillium, Aspergillus, and Alternaria. Stachybotrys chartarum (Stachybotrys atra) tends to grow on material with a high cellulose and low nitrogen content, such as fiberboard, gypsum board, paper, dust, and lint. Growth occurs when there is moisture from water damage, excessive humidity, water leaks, condensation, water infiltration, or flooding. Concern over the health effects of mold and the sensitivity of some individuals has created a need for new mold remediation methodologies apart from removal and destruction of all contaminated building material.

What is needed is a process and apparatus that permits the microbicidal properties of chlorine dioxide gas to be made available to individuals and businesses for microbial decontamination including against bacteria, bacterial spores, viruses and molds.

### BRIEF SUMMARY OF THE INVENTION

The present invention provides simplified methods and apparatus for the generation of chlorine dioxide gas in sufficient quantities and for a sufficient time to result in microbial decontamination of enclosed spaces including rooms, dwellings, buildings, containers and transportation vessels.

A process is provided for generating chlorine dioxide that includes adding reactants for generation of chlorine dioxide by dropwise addition to an aqueous reaction medium in a sealed reaction chamber while bubbling a motive gas through the aqueous reaction medium, whereby the motive gas releases the generated chlorine dioxide gas from the aqueous reaction medium and drives the release of chlorine dioxide gas from reaction chamber. In one embodiment the reactants are added by gravity feed from reactant containers affixed or in fluid communication with the reaction chamber. Alternatively, the reactants are added by metering pumps supplied by chemical tanks.

In one embodiment of the invention, the chlorine dioxide generating reaction involves dropwise addition of aqueous solutions of sodium chlorite and hydrochloric acid to a reaction medium consisting essentially of water. In one embodiment, the sodium chlorite and hydrochloric acid are added to the aqueous reaction medium at a final weight to weight ratio of chlorite to acid in the range of about 2 to about 3.1.

In an optional alternative chemistry, the reaction between sodium chlorite and hydrochloric acid further includes sodium hypochlorite.

In another embodiment, the chlorine dioxide generating reaction involves dropwise addition of aqueous solutions of sodium chlorate and sulfuric acid to a reaction medium consisting essentially of water. Such reactions further include either sodium chloride or hydrogen peroxide.

In one embodiment of the invention, a process for generating chlorine dioxide is provided that includes providing an aqueous solution of a first reactant for generation of chlorine dioxide in a sealed reaction chamber, bubbling a motive gas through the aqueous solution, adding by dropwise addition a second reactant for generation of chlorine dioxide to the aqueous solution, whereby chlorine dioxide gas is generated and is released from the aqueous solution by the action of the motive gas which also serves to drive the release of chlorine dioxide gas from reaction chamber. The second reactant can be added either by gravity feed or by a metering pump. In this process, the first reactant for generation of chlorine dioxide is selected from the group consisting of sodium chlorite, sodium chlorate, sodium hypochlorite, hydrogen peroxide, hydrochloric acid and sulfuric acid.

In one embodiment, a process for generating chlorine dioxide includes providing a sealed reaction chamber comprising a feed port for addition of chemical reactants, a port for addition of a motive gas, and a chlorine dioxide exit port. An aqueous reaction medium is introduced into the sealed reaction chamber and a motive gas is bubbled through the aqueous reaction medium. Chemical reactants for chlorine dioxide generation are added to the aqueous reaction medium, resulting in the generation of chlorine dioxide in the aqueous reaction medium. The motive gas provides for mixing of the chemical reactants and serves to release the chlorine dioxide from the aqueous reaction medium and drive the release of chlorine dioxide gas from reaction chamber. The chemical reactants can be added by gravity feed or by a metering pump. In one embodiment of this process, the aqueous reaction medium consists essentially of water and the chemical reactants comprise aqueous solutions of sodium chlorite and hydrochloric acid. In one preferred embodiment, the aqueous solutions of sodium chlorite and hydrochloric acid are added to the aqueous reaction medium at a final weight to weight ratio of chlorite to acid in the range of about 2 to about 3.1.

In alternate chemistries according to the methods and apparatus of the invention, the chemical reactants include sodium chlorite and hydrochloric acid and sodium hypochlorite. Where the source of the Cl⁻ ion is sodium chlorate, chlorine dioxide can be formed by reaction with sulfuric acid and sodium chloride or hydrogen peroxide.

In one embodiment, a chlorine dioxide generator is provided that includes a sealable reaction chamber dimensioned to contain an aqueous reaction medium, one or more chemical feed ports for dropwise addition of chemical reactants to the aqueous reaction medium, one or more motive gas conduits arrayed for bubbling a motive gas through the reaction medium, and one or more chlorine dioxide exit ports. The generator may optionally include one or more reactant containers affixed to the reaction chamber for introduction of the chemical reactants by gravity feed. Alternatively, the generator may include one or more chemical feed tanks in fluid communication with the chemical feed ports. The feed tanks may be connected to the reaction chamber via one or more metering pumps for a controlled feed of chemical reactants to the aqueous reaction medium.

In one embodiment of the chlorine dioxide gas generator of the invention, a source of compressed gas in gaseous communication with the motive gas conduit is provided in the form of a gas compressor or, alternatively, a tank of compressed gas.

The generator may optionally be skid mounted and the skid may include other equipment including chemical feed tanks and metering pumps.

In one embodiment, apparatus and methods are provided for the generation of chlorine dioxide in a sealed reaction chamber, the method including acidifying a sodium chlorite solution with hydrochloric acid by slow addition of the sodium chloride solution and the hydrochloric acid to an aqueous reaction medium in the presence of a motive stream of gas bubbled through the reaction medium, wherein the motive gas catalyses the release of chlorine dioxide from the reaction medium while simultaneously increasing the atmospheric pressure within the reaction chamber to induce a steady flow of chlorine dioxide gas from the chamber. In one embodiment utilizing this chemistry, the sodium chlorite and hydrochloric acid are added to the aqueous reaction medium at a final weight to weight ratio of chlorite to acid in the range of about 2 to about 3.1.

In one embodiment, a novel method for remediation of microbial contamination in an enclosed space is provided using in situ generation of chlorine dioxide gas. In accordance with this embodiment, a chlorine dioxide gas reaction chamber is placed in gaseous communication with the interior of the contaminated space, an aqueous reaction medium is added to the chlorine dioxide gas reaction chamber and a stream of motive gas is bubbled through the reaction medium. Chlorine dioxide gas is generated in the reaction chamber by the dropwise addition of reactants for chlorine dioxide generation to the reaction medium and chlorine dioxide gas is released from the reaction chamber using the motive gas stream for a period of time sufficient to reduce a level of microorganisms in the enclosed space. The method is applicable to the remediation of contamination with microorganisms including mold, bacteria, bacterial spores, and viruses.

### DESCRIPTION THE DRAWINGS

FIG. 1 is a diagram of a cross sectional side of one embodiment of a chlorine dioxide generator according to the invention.

FIG. 2 is a diagram of a top view of one embodiment of a chlorine dioxide generator according to the invention.

FIG. 3 is a cross sectional diagram of an alternate metering pump embodiment of a chlorine dioxide generator according to the invention.

FIG. 4 is a diagram of a top view of an alternate metering pump embodiment of a chlorine dioxide generator according to the invention.

### DETAILED DESCRIPTION OF THE INVENTION

Simple methods and apparatus for the generation of chlorine dioxide gas are provided making the method applicable to the direct fumigation of enclosures including living spaces. Chlorine dioxide gas is generated on site and released directly into the enclosure.

Due to its potential for explosive decomposition at high concentration, chlorine dioxide is most typically entrained in a motive stream of water as soon as it is produced. Therefore chlorine dioxide is rarely used in gaseous form for fumigation of large enclosures and spaces. In one newly developed method for utilizing chlorine dioxide as a fumigant, the chlorine dioxide is generated and immediately entrained in water as is typical for water treatment. The water is then run over a packed bed column and the chlorine dioxide released by a stream of air flowing over the bed. This method is expensive and technically difficult thus limiting the applicability of chlorine dioxide fumigation except for extreme emergencies.

The present invention provides a novel chlorine dioxide generator in which the chlorine dioxide is produced in an aqueous reaction medium as a partially dissolved gas. In the method and apparatus of the present invention, reactants are combined by aqueous addition in a closed reaction chamber into which a motive stream of air or other non-reactive gas is introduced under pressure beneath the surface of an aqueous reaction medium. The bubbles of motive gas rise naturally to the surface of the aqueous solution. The action of the bubbles provides a motive force for the mixing of reactants and for the release and movement of chlorine dioxide gas as it is generated to the surface of the aqueous solution in the reaction chamber. As a consequence of the flow of motive gas into the reaction chamber, the chlorine dioxide is continuously diluted and displaced from the reaction chamber by gas pressure. By virtue of the contemporaneous production and continuous release of the chlorine dioxide, explosive concentrations can be avoided while producing the gas in a directly usable form. As a consequence, the chlorine dioxide can be safely generated in gaseous form and is directly available for fumigation as soon as it exits the reaction chamber. In addition, because of the bubbling action, the chlorine dioxide gas leaves the chamber in a humidified cloud.

Reactants for chlorine dioxide generation may be added to the aqueous reaction medium either by metered addition or by gravity feed from reactant containers affixed to the reaction chamber. Gravity fed drip methods may include utilizing aperture dimension to control the rate of addition depending on the hydrostatic pressure, concentration and viscosity of reactant solutions. Alternatively, spigots or valves may be employed.

Compressed motive gas is introduced into the chamber through a conduit having an outlet under the surface of the aqueous reaction medium. By the term “motive gas”, it is meant a standard mixture of atmospheric gases (“air”) as well as other gases that provide a motive force for the release of chlorine dioxide gas in aqueous solution. Gases such as for example nitrogen, or gas mixtures may alternatively be used if not chemically interfering with the chlorine dioxide generation reaction. Where the term “air” is used, this is to provide a convenient distinction from chlorine dioxide gas and is not intended as a limitation as to chemical composition.

As the motive gas enters the reaction chamber under the surface of an aqueous solution in the bottom of the chamber and rises to the surface of the aqueous solution, the gas provides a motive force for release of chlorine dioxide gas from the aqueous solution in which it is formed. The released chlorine dioxide gas rises to the top of the chamber where it exits through one or more exit ports in the top of the chamber. The steady introduction of motive gas into the sealed reaction chamber results in an increase in pressure within the chamber. This pressure together with the provision of the exits ports produces an exit stream that carries the generated chlorine dioxide gas out of the chamber as it is produced. The motive gas such as compressed air can be supplied from any suitable means including for example an air compressor or compressed air tank. Where a compressed air tank is used, the tank can optionally be secured to the outside of the reaction chamber. If desired, the tank may be fitted with a regulator valve for constant steady release of motive gas into the chamber. The rate at which motive gas is introduced into the chamber establishes the rate at which chlorine dioxide gas exits the chamber and may be tuned as desired, while insuring that the rate is sufficient to avoid the build-up of explosive concentrations of chlorine dioxide within the chamber.

Depending on the configuration of the chemical feed ports, different chemical microenvironments can form in aqueous solution. The presence and/or chemical characteristics of these microenvironments can be manipulated by the combined configuration of the chemical inlet ports and the motive gas introduction port. If desired, a plurality of motive gas release outlets can be disposed around the bottom of the reaction chamber to “tune” the mixing of chemical reactants and release of dissolved chlorine dioxide gas.

Several different chemistries may be employed to generate chlorine dioxide gas in accordance with the methods and apparatus provided herein. Several reaction chemistries starting with sodium chlorite, NaClO₂, can be performed using the process and apparatus of the present invention including, for example, the acidification of sodium chlorite (NaClO₂) with hydrochloric acid (HCl) or the acidification of both sodium chlorite (NaClO₂) and sodium hypochlorite (NaOCl) with hydrochloric acid (HCl).

Likewise, several reaction chemistries starting with sodium chlorate, NaClO₃, can be performed using the process and apparatus of the present invention including, for example, the reduction of sodium chlorate (NaClO₃) with sulfuric acid (H₂SO₄) in the presence of either sodium chloride (NaCl) or hydrogen peroxide (H₂O₂).

The invention is not limited to a combination of two or three reactants in water. Alternatively, the reaction chamber can contain an aqueous solution of one reactant to which the other reactant is added by dropwise addition. For example, a desired volume of an aqueous solution of HCl can be placed in the reaction chamber and NaClO₂ can be added by dropwise addition in the presence of a motive gas bubbling through the HCl solution.

The reaction chamber and fittings can be constructed of any non-reactive material including certain stainless steels and non-reactive exotic alloys. Preferably, non-metallic materials are employed such as polyvinyl chloride (“PVC”), chlorinated polyvinyl chloride (“CPVC”), polyethylene (“PE”), crosslinked polyethylene (“PEX”), and various fluoropolymers including for example polyvinylidene fluoride (“PVDF”, such as KYLAR brand PVDF) and polytetrafluoroethane (“PTFE”, such as DuPont TEFLON brand PTFE).

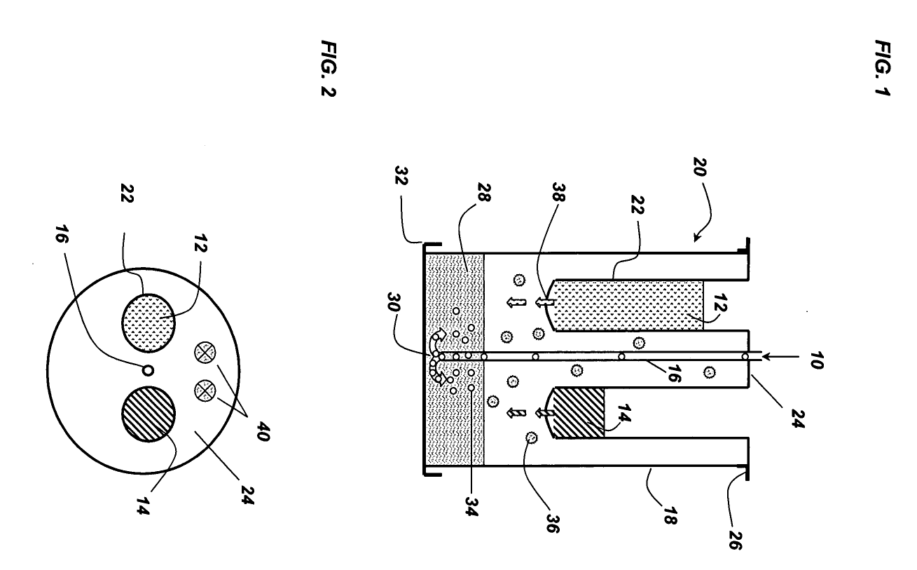

In one embodiment, an apparatus is provided for producing chlorine dioxide gas by gravity fed reactant addition as generally depicted in FIG. 1. Reaction chambers of different dimensions can be readily constructed from commercially available materials such as PVC pipe. For example, a reaction chamber 20 may constructed out of a 28 inch section of 12 inch PVC pipe 18. The bottom of pipe 18 is permanently closed with cap 32. A 12 inch PVC flange collar 26 is chemically welded around the top of pipe 18. A 12 inch PVC blind flange 24 forms the top of the reaction chamber. The top can be affixed to the reactant chamber by bolts, clips, clamps and the like during operation. Reactant containers 22 may be formed from bottom capped PVC pipes such as, for example, 17 inch long, 4 inch diameter, PVC pipes mounted to, and extending downwardly from, top blind flange 24. A small aperture 38 is formed in each bottom cap for the controlled release of reactants. A reaction chamber produced in accordance with the above description will readily accommodate approximately 1-2 gallons (3.78-7.6 liters) of aqueous reaction medium with a total fluid capacity in the reaction chamber up to the bottom of the reactant containers 22 of approximately 5.6 gallons (21.2 liters).

This design is suitable for both two and three component reactions. In a two component reaction such as, for example, between sodium chlorite (NaClO₂) and hydrochloric acid (HCl), the top flange could have two 4 inch PVC cylinders attached, one cylinder 22 containing a measured quantity of an HCl solution 14 and the other cylinder containing a measured quantity of an aqueous NaClO₂ solution 12.

Where reaction chemistries require disproportionate volumes of the reactants, a further cylinder can be added to permit the use of a greater volume of one reactant. Alternatively, use of cylinders of different diameters and thus total volume may be optionally employed. In a three component reaction such as for example between sodium chlorite, sodium hypochlorite and hydrochloric acid or between sodium chlorate, hydrogen peroxide and sulfuric acid, three 4 inch PVC cylinders can be attached to the top flange.

Motive gas introduction conduit 16 is fixedly mounted to and extends downward from the top blind flange 24. The top of the gas introduction conduit is fitted for communication with a source 10 of motive gas such as for example from an air compressor or compressed air tank. Although air is suitable, other non-reactive compressible gases are suitable as well. Motive gas introduction conduit 16 extends to the bottom of the reaction chamber. In one embodiment, motive gas introduction conduit 16 is constructed of a bottom capped ½ inch pipe tube. The bottom end cap has perforations 30 to allow the release of gas from the bottom of the reaction chamber. Any means for the generation of bubbles may be employed including one or more small perforations, sparge stones and the like. Alternatively, the motive gas can enter the chamber through a port formed in the bottom of the chamber.

A top view of the top blind flange is depicted in FIG. 2. As depicted on FIG. 2, the top flange also has one or, preferably two, ports 40 for release of the chlorine dioxide from the chamber. In one embodiment, the ports are two ¾ inch PVC threaded nipples fitted with valves for releasing the chlorine dioxide gas.

FIG. 1 also provides a depiction of an apparatus and method in operation for a two component reaction such as between NaClO₂ and HCl in water as the aqueous reaction medium. Water 28 is placed in the bottom of the reaction chamber 20. Top 24 is bolted into place. Measured quantities of reactants are placed in each reactant container 22 depending on the desired final concentration of chlorine dioxide relative to the volume of space to be treated. As depicted in FIG. 1, an aqueous solution of sodium chlorite 12 is placed in one reactant container. An aqueous solution of HCl 14 is placed in the other reactant container. Reactants begin to drip from these containers through bottom apertures 38. Motive gas is released into the bottom of reaction chamber through bottom end cap 30 and increases the atmospheric pressure within the reaction chamber. As chlorine dioxide is generated, the motive force of the bubbles 34 releases chlorine dioxide gas 36 from the water. The chlorine dioxide gas in humidified solution is diluted with the motive gas and forced by the increased pressure to exit the reaction chamber through top valves 40. The chlorine dioxide gas leaves the reaction chamber as a humidified fog of fumigant gas.

The reaction is permissive to some variation in reactant concentrations. A pH range of about 1.9 to about 2.6 and a humidity of over 70% is preferable. For example, in laboratory tests chlorine dioxide gas was effectively produced from the dropwise addition of NaClO₂ and HCl to water at final chlorite to acid weight ratios in the range of about 2 to about 3.1 over an almost 10 fold range in the volume ratio of reactants to water. A pH of 2.0 was obtained with a mixture of 11 gm of 25% NaClO₂ and 3.7 gm of 37% HCl in 300 ml of water and having an approximate weight ratio of chlorite to acid of about 2. A pH of 2.5 was obtained with a mixture of 1.6 gm of 25% NaClO₂ and 0.4 gm of 37% HCl in 300 ml of water and having an approximate chlorite to acid weight ratio of about 2.7. Preferably, a weight ratio centered around approximately 2 to about 3.1 is employed in accordance with the following calculations for a two component reaction between sodium chlorite and hydrochloric acid as follows:
5 NaClO₂+4 HCl→4 ClO₂+5 NaCl+2H₂O

The reaction was calculated utilizing the values set out below:

| Reaction | | | | | |
|---|---|---|---|---|---|
| Chemistry | 5 NaClO₂ | 4 HCl | 4 ClO₂ | 5 NaCl | 2H₂O |
| Molecular | 5 (90.44) | 4 (36.46) | 4 (67.45) | 5 (58.45) | 2 (18) |
| weight | | | | | |
| of constituents | | | | | |
| Total MW | 452 | 146 | 270 | 292 | 36 |

452/146 = ˜3.1 (weight ratio of chlorite/acid)

The reaction can be scaled up to produce sufficient chlorine dioxide gas for the fumigation of room sized enclosures. For example, to treat a 1600 cubic foot space, approximately one gallon of water is placed in the reaction chamber and the reaction chamber sealed. A motive gas stream of compressed air is initiated. Approximately 200 ml of 31% HCl is added to one reactant cylinder and approximately 500 ml of 31% NaClO₂ is added to the other reactant cylinder to provide an approximated weight ratio of about 2.6 chlorite to acid. The molar ratio of reactants is approximately 1:1. The operator leaves the space, closing as many openings as possible. Reactants drip slowly into the water and eventually begin to produce chlorine dioxide gas, which is then continuously produced over a prolonged period of time until reactants run out and the generated chlorine dioxide gas is exhausted from the reaction chamber. After approximately 24 hours the space can be reentered.

For fumigation of larger enclosures, particularly having a number of connected rooms, such as homes, buildings, or ships, etc. that have forced air movement systems such as fan driven air handlers on the heating and air conditioning systems, the fans may be turned on to continuous operation during the fumigation process to continually recirculate the chlorine dioxide gas throughout the desired space. The quantity of reactants placed in the reactant containers is increased proportionately depending on the desired ppm of chlorine dioxide gas respective to the volume of space to be treated. Chlorine dioxide gas in the range of 300-1000 ppm is desirable for fumigation purposes. Assuming a reaction efficiency of ˜80%, chlorine dioxide gas in a concentration of approximately 600-1000 ppm in a 30,400 cubic foot structure (3800 square foot structure with 8 foot ceilings) is calculated to be obtained by the reaction of approximately 5.6 liters of 31% sodium chlorite and 2.2 liters of 31% HCl. The quantity of chlorine dioxide gas in this total volume is calculated to be approximately 1.7 mg/liter, well below the potentially explosive 300 mg/liter.

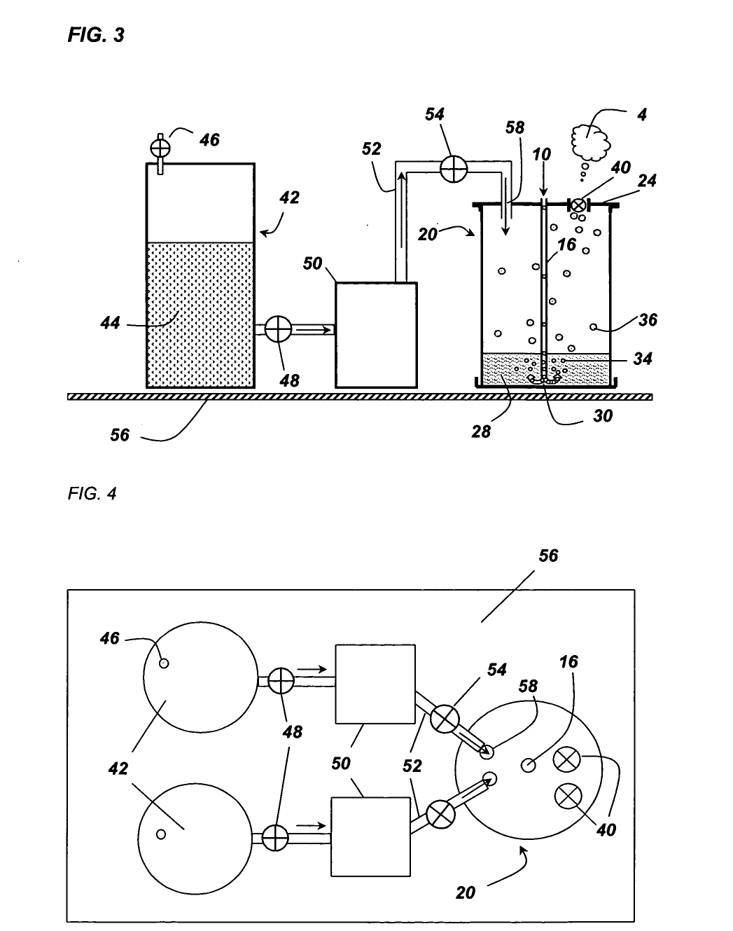

In an alternate embodiment, a skid mounted chlorine dioxide generator is provided. Referring to FIGS. 3 and 4, which provide side and top views respectively, of one example of a skid mounted generator apparatus, reactants are provided in chemical feed tanks 42, each of which may be fitted with a vent 46 if need be. The reactants enter metering pumps 50 through block valve 48. The metering pumps 50 are connected to reaction chamber 20 through conduit lines 52 and block valves 54 and enter the reaction chamber through ports 58. Any or all of the feed tanks, pumps, reaction chamber, and any ancillary supplies and equipment can be mounted on skid 56.

This design is suitable for both two and three component reactions. In a two component reaction such that depicted, two chemical feed tanks 42, one for each separate reactant, are provided, as well was two metering pumps 50 and related conduits, valves and ports. In a three component reaction such as for example between sodium chlorite, sodium hypochlorite and hydrochloric acid or between sodium chlorate, hydrogen peroxide and sulfuric acid, three feed tanks, metering pumps and so forth are mounted on the skid.

Prior to initiating the addition of reactants, aqueous medium 28 is added to reaction chamber 20. For long running reactions, further water can be added through the provision of a further port and associated metering pump in fluid communication with the reaction chamber such that the volume available in the reaction chamber is not limiting. Motive gas as provided from source 10 from an air compressor or compressed air tank and enters the reaction chamber through perforations 30 at the bottom of air conduit 16. As the generation of chlorine dioxide begins with metered addition of reactants, raising motive gas bubbles 34 provide a motive force for the mixing of reactants and for the liberation of chlorine dioxide gas from the aqueous solution. The humidified chlorine dioxide gas 4 exits the chamber through valve 40 and enters the space to be fumigated. A skid mounted chlorine dioxide generator provides for remote operation. In addition, the duration of time that the skid mounted generator runs and thus the amount of chlorine dioxide generated is only limited by the volume of reactants provided by metered addition. Metered addition of reactants provides for precise control of reactant addition in terms of both volume and volume per time. The present metered addition embodiment is suitable for the prolonged fumigation of large spaces and may be optionally combined with fans or blowers or may be placed in communication with duct work.

The invention is suitable for the fumigation of a variety of spaces including, for example, buildings, dwellings, ships, grain elevators and containers such as shipping containers. Standard dimensions for dry cargo containers are approximately 20′×8′×8′ (approximately 1280 cubic feet) to 40′×8′×8′ (approximately 2560 cubic feet). Larger standard containers having greater height or length have a cubic foot capacity of up to 3,000 cubic feet. The fumigation can be used to treat enclosures contaminated with a variety of organisms including bacteria, bacterial spores, viruses, molds, and insects.

While the invention has been disclosed with respect to a limited number of embodiments, numerous modifications and variations will be appreciated by those skilled in the art. It is intended, therefore, that the following claims cover all such modifications and variations that may fall within the true spirit and scope of the invention.

1. A process for generating chlorine dioxide comprising:
adding reactants for generation of chlorine dioxide by dropwise addition to an aqueous reaction medium in a sealed reaction chamber while bubbling a motive gas through the aqueous reaction medium, whereby the motive gas releases the generated chlorine dioxide gas from the aqueous reaction medium and drives the release of chlorine dioxide gas from reaction chamber.

2. The process of claim 1, wherein the reactants are added by gravity feed.

3. The process of claim 1, wherein the reactants are added by a metering pump.

4. The process of claim 1, wherein the aqueous reaction medium consists essentially of water and the reactants comprise sodium chlorite and hydrochloric acid.

5. The process of claim 4, wherein the sodium chlorite and hydrochloric acid are added to the aqueous reaction medium at a final weight to weight ratio of chlorite to acid in the range of about 2 to about 3.1.

6. The process of claim 4, wherein the reactants further comprise sodium hypochlorite.

7. The process of claim 1, wherein the aqueous reaction medium consists essentially of water and the reactants comprise sodium chlorate and sulfuric acid.

8. The process of claim 7, wherein the reactants further comprise either sodium chloride or hydrogen peroxide.

9. A process for generating chlorine dioxide comprising:
providing an aqueous solution of a first reactant for generation of chlorine dioxide in a sealed reaction chamber;
bubbling a motive gas through the aqueous solution;
adding a second reactant for generation of chlorine dioxide to the aqueous solution; whereby the motive gas releases the generated chlorine dioxide gas from the aqueous solution and drives the release of chlorine dioxide gas from reaction chamber.

10. The process of claim 9, wherein the second reactant is added by gravity feed.

11. The process of claim 9, wherein the second reactant is added by a metering pump.

12. The process of claim 9, wherein the first reactant for generation of chlorine dioxide is selected from the group consisting of: sodium chlorite, sodium chlorate, sodium hypochlorite, hydrogen peroxide, hydrochloric acid, and sulfuric acid.

13. A process for generating chlorine dioxide comprising:
providing a sealed reaction chamber comprising a feed port for addition of chemical reactants, a port for addition of a motive gas, and a chlorine dioxide exit port;
introducing an aqueous reaction medium into the sealed reaction chamber;
bubbling the motive gas through the aqueous reaction medium; and
feeding the chemical reactants to the aqueous reaction medium, whereby chlorine dioxide is generated in the aqueous reaction medium and the motive gas releases the chlorine dioxide from the aqueous reaction medium and drives the release of chlorine dioxide gas from reaction chamber.

14. The process of claim 13, wherein the chemical reactants are added by gravity feed.

15. The process of claim 13, wherein the chemical reactants are added by a metering pump.

16. The process of claim 13, wherein the aqueous reaction medium consists essentially of water and the chemical reactants comprise aqueous solutions of sodium chlorite and hydrochloric acid.

17. The process of claim 16, wherein the aqueous solutions of sodium chlorite and hydrochloric acid are added to the aqueous reaction medium at a final weight to weight ratio of chlorite to acid in the range of about 2 to about 3.1.

18. The process of claim 16, wherein the chemical reactants further comprise sodium hypochlorite.

19. The process of claim 13, wherein the aqueous reaction medium consists essentially of water and the chemical reactants comprise sodium chlorate and sulfuric acid.

20. The process of claim 19, wherein the chemical reactants further comprise a chemical selected from the group consisting of: sodium chloride and hydrogen peroxide.

21. A chlorine dioxide generator comprising:
a sealable reaction chamber dimensioned to contain an aqueous reaction medium;
one or more chemical feed ports in the reaction chamber for dropwise addition of chemical reactants to the aqueous reaction medium;
one or more motive gas conduits in the reaction chamber and arrayed for bubbling a motive gas through the reaction medium; and
one or more chlorine dioxide exit ports in the reaction chamber for exhaust of chlorine dioxide generated by interaction between the chemical reactants in the aqueous reaction medium and released by the action of the motive gas.

22. The generator of claim 21, further comprising one or more reactant containers affixed to the reaction chamber for introduction of the chemical reactants by gravity feed.

23. The generator of claim 21, further comprising one or more chemical feed tanks in fluid communication with the chemical feed ports.

24. The generator of claim 23, further comprising one or more metering pumps for a controlled feed of chemical reactants to the aqueous reaction medium.

25. The generator of claim 21, further comprising a source of compressed gas in gaseous communication with the motive gas conduit.

26. The generator of claim 21, further comprising a skid for mounting of the reaction chamber.

27. A method for the generation of chlorine dioxide in a sealed reaction chamber, comprising acidifying a sodium chlorite solution with hydrochloric acid by slow addition of the sodium chloride solution and the hydrochloric acid to an aqueous reaction medium in the presence of a motive stream of gas bubbled through the reaction medium, wherein the motive gas catalyses the release of chlorine dioxide from the reaction medium while simultaneously increasing the atmospheric pressure within the reaction chamber to induce a steady flow of chlorine dioxide gas from the chamber.

28. The method of claim 27, wherein the sodium chlorite and hydrochloric acid are added to the aqueous reaction medium at a final weight to weight ratio of chlorite to acid in the range of about 2 to about 3.1.

29. A method for remediation of microbial contamination in an enclosed space comprising: placing a chlorine dioxide gas reaction chamber in gaseous communication with the interior of the enclosed space;
adding an aqueous reaction medium to the chlorine dioxide gas reaction chamber;
bubbling a motive stream of gas through the reaction medium;
generating chlorine dioxide gas in the reaction chamber by the dropwise addition of chlorine dioxide generating reactants to the reaction medium;
releasing chlorine dioxide gas from the reaction chamber using the motive gas stream for a period of time sufficient to reduce a level of microorganisms in the enclosed space.

30. The method of claim 29, wherein the microorganisms are selected from the group consisting of: mold, bacteria, bacterial spores, and viruses.
